(19) United States
(12) Patent Application Publication
(10) Pub. No.: US 2009/0156840 A1
(43) Pub. Date: Jun. 18, 2009

(54) Production of cyclic diesters of alpha-hydroxyacids

(76) Inventor: Samuel Joseph Wajc, Haifa (IL)

Correspondence Address: WAJC Samuel Joseph, Rehov Denya 48, Haifa, 34980, IL

(21) Appl. No.: 12/002,218

(22) Filed: Dec. 17, 2007

(51) Int. Cl. C07D 319/12

(52) U.S. Cl. 549/379

(57) ABSTRACT

The raw material used in most process for the production of cyclic diesters of alpha-hydroxyacids, such as Lactide and Glycolide, is a 10% solution of Hydroxyacid. During the evacuation of the solution and condensation reaction water, the organic molecules may be damaged.

The present process is characterized by the use of reactants that bring as little water as possible to the reactor. In particular, for the production of Lactide, the main reactant may be anhydrous Calcium Lactate, that will react at about room temperature, for instance, with Sulfuric Anhydride, to give a solid mixture of Lactide, Calcium Sulfate Dihydrate and Calcium Sulfate Hemihydrate. Partial dehydration of the Dihydrate under mild temperature is followed by distillation of the Lactide out of the remaining powder of Calcium Sulfate Hemihydrate, and desublimation of the Lactide. Purification of the Lactide is done in situ in the desublimator.

### US PATENT REFERENCES

U.S. Pat. No. 3,429,777 Mar. 1, 1965 Bode, Herman
U.S. Pat. No. 5,319,107 Mar. 19, 1992 Benecke, Herman et al.
U.S. Pat. No. 5,357,035 Oct. 18, 1994 Gruber, Patrick et al.
U.S. Pat. No. 5,766,439 Jun. 16, 1998 Eyal, Aharon et al.
U.S. Pat. No. 6,800,767 Aug. 4, 2003 Coszach, Philippe et al.
US Pat Application 20050281913 Dec. 22, 2005 Van Krieken, Jan et al.
Provisional Pat. Appl. 60/874,475 Dec. 13, 2006 Wajc, Samuel

### OTHER REFERENCES

Yukoho Sakata et al., 2005 (“Characterization of dehydration and hydration behaviour of calcium lactate pentahydrate and its anhydrate”, 2005, Colloids and Surfaces B: Biointerfaces, v. 46, pp. 135-141)
Chipera, Steve et al., 2007 (“Experimental stability of magnesium sulfate hydrates that may be present on Mars”, 2007, Geochimica et Cosmochimica Acta, v. 71, pp. 241-250)

### BACKGROUND OF THE INVENTION

This Patent Application continues the Provisional Patent Application US60/874,475 with the same title.

A key intermediate in the production of polylactic acid (PLA) is the cyclic diester Lactide (LD).

After purification of the LD to rather stringent requirements, polymerization up to molecular weight of 400 000 by ring-opening in the presence of a homogeneous catalyst is relatively easy (U.S. Pat. No. 5,319,107).

The production of LD itself by the classical process is more intricate:

a.—fermentation of a well-chosen carbohydrate in the presence of Ca(OH)₂ (or CaCO₃) leads to the production of a suspension of bacteria in a solution of Calcium Lactate (CaLac).
b.—the bacteria are separated by centrifugation or filtration and discarded (U.S. Pat. No. 5,766,439)
c.—the filtrate reacts with Sulfuric Acid, which causes the precipitation of Gypsum (Calcium Sulfate Dihydrate) and the liberation of Lactic Acid (LA) as a solution of some 10% by weight. (US Pat. Appl. No. 20050281913)
d.—that solution is concentrated by distillation to 85-88% LA by weight.
e.—the concentrated LA solution, in the presence of a homogeneous catalyst, undergoes a prepolymerization in a vacuum distillation column, where more water is separated.
f.—as the molecular weight of the prepolymer is only about 1000, its mechanical properties are not suitable for industrial use.
g.—the prepolymer is then depolymerized by back-biting in the presence of a catalyst under vacuum and the LD leaving the reactor in the vapor phase is condensed as a liquid or directly fed to a distillation column to produce liquid crude LD (mixed with LA, some of its light oligomers, water, unwanted enantiomers of LD, etc. . . ) (U.S. Pat. No. 5,357,035).
h.—the crude LD is further purified by liquid-liquid extraction with water followed by crystallization from aqueous solution (US Pat. Appl. No. 20060014975).
i.—centrifugation gives a cake of purified LD, but since the impurity level is still too large, a last operation is required:
j.—melt crystallization with sweating to remove the impurities by gravity flow.

All these operations are well known, so that it is possible to produce for instance the enantiomer L-LD with a purity of up to 99.9%. But the yield of each of the operations of this long chain is rather modest, so that large recycle flows are required, so that the various equipment items tend to be large with large energy requirements.

In the classical process, a large amount of waste product (Calcium Sulfate Dihydrate, or gypsum) is produced.

Other alpha-hydroxyacids, such as glycolic acid, may similarly be dimerized to the corresponding cyclic diester and thence to the polyacid (e.g. glycolide leading to polyglycolic acid) by the same process and with the same disadvantages.

If it were possible to go from the Calcium Lactate directly to the LD, the production cost of PLA would decrease. This is the object of the present invention.

### BRIEF SUMMARY OF THE INVENTION

Since the whole classical process of LD production may be characterized as a concatenation of ever more difficult dehydration steps, and since dehydration at temperatures larger than 200° C. may lead to thermal degradation or to racemization of the LD, we have invented a process where the reactants introduced to the LD production reactor are themselves water absorbents, so that little or no water has to be evacuated, and so that by stoichiometry the only possible volatile reaction product is LD.

### DETAILED DESCRIPTION OF THE INVENTION

From the classical chain of operations, we retain only the first two ones:

A.—Fermentation of a well-chosen carbohydrate in the presence of Ca(OH)₂ (or CaCO₃) leads to the production of a suspension of bacteria in a solution of Calcium Lactate (CaLac).
B.—The bacteria are separated by centrifugation or filtration and discarded

The next three steps are similar to those disclosed in U.S. Pat. No. 5,766,439:

C.—The filtrate (or centrate), a solution of Calcium Lactate, is concentrated by evaporation of water
D.—Cooling crystallization brings about separation of a hydrate of Calcium Lactate (the pentahydrate if the crystallization temperature is low enough)
E.—Separation of the crystals by centrifugation; further treatment of the mother-liquor for separation of more CaLac Pentahydrate (CaLac PH) and separation of a bleed solution.

The following steps embody the gist of the present invention:

F.—Dehydration of the Pentahydrate at atmospheric pressure and at a temperature smaller than 150° C. to obtain anhydrous CaLac (Yukoho Sakata et al. 2005).
G.—Production of a “cement” by reaction at room temperature of the Calcium Lactate Anhydrate with a concentrated acid or anhydride chosen among Phosphoric Anhydride, Phosphoric Acid, Sulfuric Anhydride, Sulfurous Anhydride, Oleum, Sulfuric Acid. This reactant is chosen in such a way that after reaction with the anhydrous Calcium salt of the relevant alpha-hydroxyacid a mixture of solids is produced with as little as possible crystallization water. For instance, reaction of 1 mole of anhydrous CaLac with 1 mole SO₃ would lead (by stoechiometry) to a mixture of 1 mole LD,
1 mole Calcium Sulfate and 1 mole water. This suggests that the anorganic salts would be a mixture of Calcium Sulfate Dihydrate (⅓) and Calcium Sulfate Hemihydrate (⅔).
H.—Heating the solid powder or granules to a temperature sufficient for reaction and evaporation of excess water at or slightly below atmospheric pressure. For instance, Gypsum (the Dihydrate) would loose 1.5 mole water per mole of gypsum around 125° C., too low a temperature for evaporation at atmospheric pressure of any LD or LA that might have been produced. In these conditions, i.e. in the absence of free water, no LA will remain. In practice, one slowly increases the temperature, taking care that no LA but only water appears in the condensate.
I.—Sublimation (or rather distillation, since the melting point of L-LD is 98° C.) of the LD from the mixture (for instance Calcium Sulfate Hemihydrate interspersed with LD) under vacuum or in the presence of inert gas. This is done only after setting of the “cement”.
J.—Desublimation of LD as a cylindrical solid layer on the vertical tubes of a heat exchanger.
K.—Reheating this layer in order to induce “sweating” so that an impure viscous solution will be produced, but as opposed to what happens in melt crystallizers, the impurities will be evacuated by selective sublimation and not by gravity.
L.—The last step is similar to that in melt crystallization, i.e. evacuation of the purified crystals by complete melting of the layer.

It will be apparent to those skilled in the art that elimination of most of the water at a mild temperature, before production of LA and its dimerization is the key to this process, insofar as it allows the production of fairly concentrated LD dispersed in porous agglomerates of rather inert anorganic material. Lactide may well be molten, but capillarity keeps it inside the individual anorganic granules (or agglomerates of anorganic powder) if enough inert anhydrous powder keeps these granules (or agglomerates) apart. At this stage, it is possible to distill a vapour that is mainly LD, so that desublimation gives a rather pure product that may be further purified in the desublimator itself by sweating and sublimation (without having to move the product to another apparatus).

Notice that, apart from water that will be trapped by condensation after the desublimator, the waste product is now the hemihydrate of Calcium Sulfate, a waste product that may be used in the building industry.

Referring to the drawing, we see in the upper left corner the first apparatus of the plant, namely the fermentor F, fed with sugars, bacteria and nutrients in a manner known in the art. The bacteria present in the fermentation broth are separated in a decanter-centrifuge DC, the resulting mud being partly discarded and partly recycled to the fermentor.

The centrate is a fairly dilute solution (around 10%) of Calcium Lactate that is fed to an evaporator E, followed by a cooling crystallizer CC (alternatively, an evaporator-crystallizer may be used). Condensation water is discarded and the suspension of Calcium Lactate Pentahydrate is sent to a filter-centrifuge FC. The filtrate is recycled to the evaporator E, having been purified en route. The wet cake of the filter-centrifuge FC is dehydrated in the dryer DH1 and the resulting powder of CalciumLactate Anhydrate is fed to the reactor R. Gaseous Sulfuric Anhydride is fed to the same reactor, so that a mixture of LD, CaSO₄.2H₂O and CaSO₄.0.5H₂O is produced. This mixture is fed to a second dryer DH2, where the Calcium Sulfate Dihydrate is transformed in Hemihydrate (in order to limit the temperature to less than 98° C., this operation may be performed at atmospheric pressure in the presence of an inert gas or under a small vacuum). The resulting mixture is fed to a distillation apparatus DIST, operating at less than 200° C., either under vacuum or in the presence of inert gas.

The distilled or entrained vapor will deposit the LD in the desublimator DES.

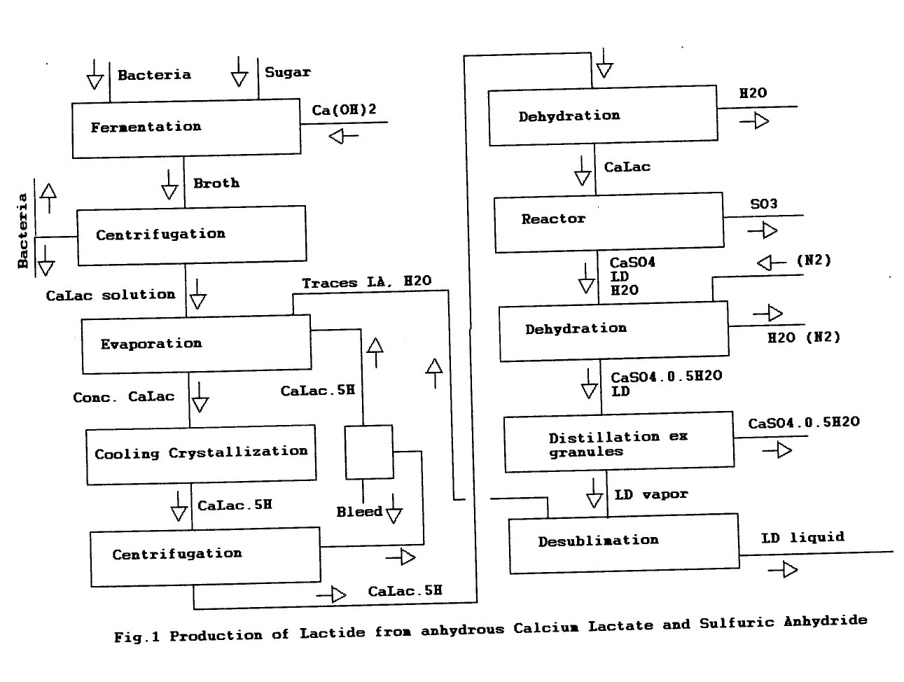

It will be apparent to those skilled in the art that Magnesium Lactate might have some advantage over Ca Lac. Its production by fermentation (U.S. Pat. No. 3,429,777), the crystallization of its Trihydrate, the dehydration of the latter and the reaction with SO₃ are as easy as those of CaLac, and after reaction, one would produce Magnesium Sulfate Monohydrate (Kieserite), whose dehydration at 160° C. is reputedly kinetically hindered, so that there would be no need for a step of partial dehydration (DH2 in FIG. 1) (Chipera, 2007).

It will also be apparent to those skilled in the art that the use of Phosphoric Anhydride (a solid at room temperature) could lead, by reaction with anhydrous CaLac, to a mixture of Monocalcium Phosphate and Dicalcium Phosphate that might be suitable as fertilizer.

It will also be apparent to those skilled in the art that less volatile cyclic diesters could still be produced by a variant of the process described here above. Indeed, if separation by distillation and desublimation of the cyclic diester from the solid residue is not economically practical, it may still be possible to extract it (solid-liquid extraction) by a suitable solvent, such as toluene, at a temperature close to the diester melting point. This would be followed by crystallization from solution, separation by centrifugation, and drying of the cake.

### BRIEF DESCRIPTION OF THE DRAWING

A carbohydrate source (e.g. sugar), calcium hydroxide and bacteria are loaded in the fermentation reactor F.

The broth leaving the fermentation reactor F is fed to the decanter centrifuge DC, where bacteria are separated. Part of the solids (bacteria) is recycled to the fermentation reactor and the rest is discarded.

The centrate leaving DC is a solution of Calcium Lactate. It is fed to an evaporator E. If it is economically justified, a small recycled flow of condensate having escaped desublimation in DES (see further) is also fed to the evaporator E.

The resulting concentrated Calcium Lactate solution is fed to the cooling crystallizer CC.

The resulting suspension of crystals of Calcium Lactate Pentahydrate in a saturated solution of Calcium Lactate is fed to a Filter Centrifuge FC. The mother-liquor is recycled to the evaporator E, having been purified en route, so that a bleed containing impurities may be discharged.

The separated crystals of Calcium Lactate Pentahydrate are heated and dehydrated in DH1.

The powder of Calcium Lactate Anhydrate is fed to the reactor R, where it reacts with gaseous Sulfuric Anhydride.

The powder leaving the reactor R is a mixture of more or less hydrated Calcium Sulfate and Lactide. Under the right conditions, it may be granulated, or be kept as powder.

This mixture is submitted to a new dehydration in DH2.

The powder (or granulated solid) leaving DH2 is a mixture of Calcium Sulfate Hemihydrate and of Lactide.

This powder is fed to a distillation apparatus, DIST, where distillation proceeds from within the powder agglomerates (or granules). This apparatus operates under vacuum or at atmospheric pressure under Nitrogen entrainement.

The residue of this distillation is Calcium Sulfate Hemihydrate, discharged as a powder (or as granules).

The vapor leaving DIST is desublimated in the desublimator DES. The crystals of Lactide collected in DES are purified in situ and discharged as a melt.

I claim:

1. A process for the synthesis of cyclic diesters of alpha-hydroxyacids, where the reactants are anhydrous alkalino-earth salts of the corresponding alpha-hydroxyacids and a member of the group Sulfinuric Anhydride, Sulfurous Anhydride, Oleum, Sulfuric Acid, Phosphoric Anhydride, Phosphoric Acid, chosen in such a way that after reaction a pasty or solid mixture is produced that, if need be, may be further partially dehydrated at a temperature that is low enough to prevent racemization or other damage to the organic product of interest, and that may then be submitted either to solid-liquid extraction or to distillation-desublimation to recuperate the cyclic diester.
